(19) United States
(12) Patent Application Publication
(10) Pub. No.: US 2026/0041457 A1
(43) Pub. Date: Feb. 12, 2026

(54) DEVICE TO ACCESS PERIPHERAL REGIONS OF LUNG FOR DIRECT VISUALISATION WITH TOOL ATTACHMENT

(71) Applicant: Boston Scientific Scimed, Inc., Maple Grove, MN (US)

(72) Inventors: Kevin Walsh, Wellesly, MA (US); Jeremy DiTullio, North Grafton, MA (US); Douglas W. Garrity, Waltham, MA (US); Tyler Noyes, Clinton, MA (US); Seamus F. O'Shaughnessy, Chelmsford, MA (US); Austin G. Johnson, Hudson, MA (US); Colby Harris, Weston, MA (US)

(73) Assignee: Boston Scientific Scimed, Inc., Maple Grove, MN (US)

(21) Appl. No.: 19/361,512

(22) Filed: Oct. 17, 2025

Related U.S. Application Data
(63) Continuation of application No. 16/875,371, filed on May 15, 2020.
(60) Provisional application No. 62/849,311, filed on May 17, 2019.
(60) Provisional application No. 62/849,649, filed on May 17, 2019.
(60) Provisional application No. 62/849,307, filed on May 17, 2019.

(51) Int. Cl. A61B 17/34; A61B 8/00; A61B 8/12; A61B 10/02; A61B 10/04; A61B 17/00; A61B 17/24; A61B 90/00; A61M 25/00

(52) U.S. Cl. CPC A61B 17/3478; A61B 8/12; A61B 8/445; A61B 8/4455; A61B 8/4461; A61B 8/4494; A61B 8/5238; A61B 8/54; A61B 10/0233; A61B 10/04; A61B 17/00234; A61B 17/24; A61B 17/3415; A61B 90/39; A61M 25/0026; A61B 2010/0208; A61B 2010/045; A61B 2017/0034; A61B 2017/00809; A61B 2017/242; A61B 2017/3413; A61B 2090/3925; A61M 2205/586

(57) ABSTRACT

The present disclosure relates generally to the field of medical devices. In particular, the present disclosure relates to modular assemblies, systems and methods for direct visualization and biopsy of peripheral pulmonary nodules. In one example, a modular assembly of the present disclosure may include a dual-lumen catheter attached to a distal end of a main body, a first lumen may extend through the main body, the first lumen contiguous with a first lumen of the dual-lumen catheter, a second lumen may extend through the main body, the second lumen contiguous with a second lumen of the dual-lumen catheter and an attachment member formed within the main body at a proximal end of the second lumen.

### RELATED APPLICATIONS

This application is a continuation of U.S. Non-Provisional application Ser. No. 16/875,371, filed May 15, 2020, which claims the benefit of priority under 35 U.S.C. § 119 to U.S. Provisional Application No. 62/849,311, filed on May 17, 2019, the entirety of which is incorporated herein by reference.

This application claims the benefit of priority under 35 U.S.C. § 119 to U.S. Provisional Patent Application No. 62/849,649, titled “Apparatus to Provide an Adjustable Mechanism for Radial Ultrasound Port and Flush Port”, filed on May 17, 2019, the entirety of which is incorporated herein by reference.

This application claims the benefit of priority under 35 U.S.C. § 119 to U.S. Provisional Patent Application No. 62/849,307, titled “Radial Ultrasound Needle Biopsy Devices”, filed on May 17, 2019, the entirety of which is incorporated herein by reference.

### FIELD

The present disclosure relates generally to the field of medical devices. In particular, the present disclosure relates to modular assemblies, systems and methods for direct visualization and biopsy of peripheral pulmonary nodules.

### BACKGROUND

Due to the complex anatomy of the pulmonary system, a complicating factor in accurately and efficiently obtaining biopsy samples from pulmonary nodules in the peripheral regions of the lung is the inability to maintain direct visualization of the target pulmonary nodule as the biopsy sample is being taken and/or while the biopsy instrument is repositioned or exchanged through a working channel of the bronchoscope.

A variety of advantageous medical outcomes may therefore be realized by the modular assemblies, systems and method of use, of the present disclosure.

### SUMMARY

In one aspect, a modular assembly of the present disclosure may include a dual-lumen catheter attached to a distal end of a main body. A first lumen may extend through the main body. The first lumen may be contiguous with a first lumen of the dual-lumen catheter. A second lumen may extend through the main body. The second lumen may be contiguous with a second lumen of the dual-lumen catheter. An attachment member may be formed within the main body at a proximal end of the second lumen.

In the described and other embodiments, the first lumens of the main body and the dual-lumen catheter may be configured to receive a first endoscopic instrument therethrough. The second lumens of the main body and the dual-lumen catheter may be configured to receive a second endoscopic instrument therethrough. A first support structure may be attached to a proximal end of the main body at a proximal end of the first lumen. A second support structure may be attached to the distal end of the main body at a distal end of the first lumen. The attachment member may be configured to receive a handle assembly of the second endoscopic instrument in a snap-lock configuration.

In another aspect, the present disclosure relates to a system comprising a modular assembly. The modular assembly may include a dual-lumen catheter attached to a distal end of a main body. A first lumen may extend through the main body. The first lumen may be contiguous with a first lumen of the dual-lumen catheter. A second lumen may extend through the main body. The second lumen may be contiguous with a second lumen of the dual-lumen catheter. An attachment member may be formed within the main body at a proximal end of the second lumen. A first endoscopic instrument may extend through the first lumens of the main body and the dual-lumen catheter. A second endoscopic instrument may extend through the second lumens of the main body and the dual-lumen catheter.

In the described and other embodiments, the first endoscopic instrument may include a radial ultrasound probe and the second endoscopic instrument may include a biopsy needle. The second endoscopic instrument may be configured to move laterally between a first and second position within the dual-lumen catheter. The attachment member may be configured to receive a handle assembly of the second endoscopic instrument in a snap-lock configuration. The handle assembly may be configured to move laterally along the main body of the modular assembly. The handle assembly may be configured move from a first position in which a distal end of the second endoscopic instrument may be disposed within the second lumen of the dual-lumen catheter, and a second position in which the distal end of the second endoscopic instrument may extend beyond a distal end of the dual-lumen catheter. The handle assembly may include a plunger configured to move the second endoscopic instrument between the first and second position. The dual-lumen catheter may be configured to extend through a working channel of a bronchoscope. The second endoscopic instrument may provide columnar support to the dual-lumen catheter. A first support structure may be attached to a proximal end of the main body at a proximal end of the first lumen. A second support structure may be attached to a distal end of the main body at a distal end of the first lumen. The first and second support structures may provide a leak-proof seal for the first lumen.

In yet another aspect, the present disclosure relates to a method that may comprise advancing a bronchoscope into a pulmonary passage of a patient. A dual-lumen catheter may be advanced through a working channel of the bronchoscope such that a distal end of the dual-lumen catheter may extend beyond the distal end of the bronchoscope. A first pulmonary nodule may be visualized with a first endoscopic instrument that may extend through a first lumen of the dual-lumen catheter. A biopsy sample of the first pulmonary nodule may be acquired with a second endoscopic instrument extending through a second lumen of the dual-lumen catheter.

In the described and other embodiments, visualizing the first pulmonary nodule and acquiring the biopsy sample of the first pulmonary nodule may occur without having to exchange the first instrument with another instrument. The method may further include removing the second endoscopic instrument from within the second lumen of the dual-lumen catheter without altering a position of the first endoscopic instrument within the pulmonary passage. The method may further include ejecting the biopsy sample from within the second endoscopic instrument. The method may further include advancing the second endoscopic instrument through the second lumen of the dual-lumen catheter. The method may further include advancing the dual-lumen catheter through the pulmonary passage to visualize a second pulmonary nodule. The method may further include acquiring a biopsy sample of the second pulmonary nodule with the second endoscopic instrument. The first pulmonary nodule may include an eccentric pulmonary nodule.

### BRIEF DESCRIPTION OF THE DRAWINGS

Non-limiting embodiments of the present disclosure are described by way of example with reference to the accompanying figures, which are schematic and not intended to be drawn to scale. In the figures, each identical or nearly identical component illustrated is typically represented by a single numeral. For purposes of clarity, not every component is labeled in every figure, nor is every component of each embodiment shown where illustration is not necessary to allow those of ordinary skill in the art to understand the disclosure. In the figures:

FIGS. 1-3 provide perspective views of a direct visualization and biopsy assembly, according to one embodiment of the present disclosure.

FIGS. 4A-4C provide perspective views of a distal portion of a direct visualization and biopsy assembly, according to one embodiment of the present disclosure.

FIG. 5 provides a schematic illustration of direct visualization and biopsy assembly acquiring a biopsy sample, according to one embodiment of the present disclosure.

### DETAILED DESCRIPTION

The present disclosure is not limited to the particular embodiments described herein. The terminology used herein is for the purpose of describing particular embodiments only, and is not intended to be limiting beyond the scope of the appended claims. Unless otherwise defined, all technical terms used herein have the same meaning as commonly understood by one of ordinary skill in the art to which the disclosure belongs.

Although embodiments of the present disclosure are described with specific reference to modular assemblies, systems and methods designed to provide dual-function visualization and diagnostic sampling of pulmonary nodules within peripheral regions of the lung, it should be appreciated that such assemblies, systems and methods may be used to visualize and manipulate a variety of tissues within a variety of different body lumens and/or body passages.

As used herein, the singular forms “a,” “an,” and “the” are intended to include the plural forms as well, unless the context clearly indicates otherwise. It will be further understood that the terms “comprises” and/or “comprising,” or “includes” and/or “including” when used herein, specify the presence of stated features, regions, steps elements and/or components, but do not preclude the presence or addition of one or more other features, regions, integers, steps, operations, elements, components and/or groups thereof.

As used herein, the term “distal” refers to the end farthest away from the medical professional when introducing a device into a patient, while the term “proximal” refers to the end closest to the medical professional when introducing a device into a patient.

In various embodiments, the present disclosure relates generally to a direct visualization and biopsy modular assembly (e.g., modular tool attachment) which may include an ergonomic handle and catheter configured for dual-function use during an endoscopy procedure. By way of non-limiting example, the modular assembly may be configured for use within a bronchoscope working channel to provide real-time visualization (e.g., radial ultrasound imaging) and manipulation (e.g., diagnostic biopsy sampling) of solitary pulmonary nodules in peripheral regions of the lung. As disclosed herein, in various embodiments, the modular assembly may be configured to reposition and/or exchange an endoscopic tool/instrument (e.g., a second endoscopic instrument) within a peripheral region of the lung while maintaining direct visualization of the pulmonary nodule (e.g., with a first endoscopic instrument). In addition, or alternatively, the modular assembly may be configured to allow a medical professional to access, lock and/or manipulate an endoscopic tool/instrument attached thereto using a single hand.

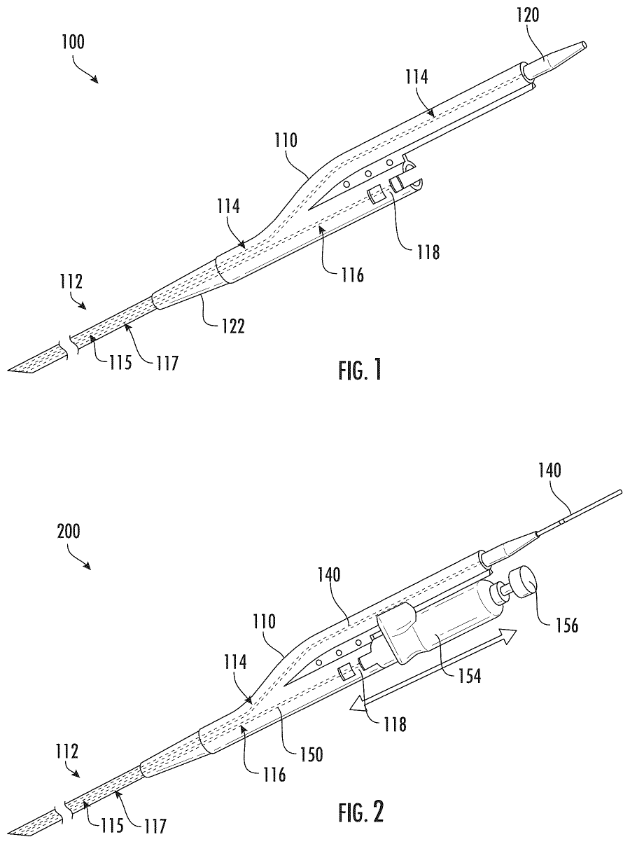

Referring to FIG. 1, in one embodiment, a modular assembly 100 of the present disclosure may include a flexible dual-lumen catheter 112 (e.g., dual-lumen elongate shaft, etc.) attached to or otherwise extending from a proximal end of a main body 110 (e.g., housing, ergonomic handle, etc.). First and second lumens 114, 116 may extend through the main body 110 such that the first lumen 114 is contiguous (e.g., coextensive) with a first lumen 115 of the dual-lumen catheter 112, and the second lumen 116 is contiguous with a second lumen 117 of the dual-lumen catheter 112. In various embodiments, the contiguous first lumens 114, 115 of the main body 110 and dual-lumen catheter 112 may be configured to receive a first endoscopic instrument 140 (e.g., a radial ultrasound probe) therethrough, e.g., in a rotatable fashion, and the contiguous second lumens 116, 117 of the main body 110 and dual-lumen catheter 112 may be configured to slidably and/or rotatably receive a second endoscopic instrument 150 (e.g., a biopsy needle) therethrough. An attachment member 118 may be formed within or otherwise attached to the main body 110 at or adjacent to a proximal end of the second lumen 116. As discussed below, in various embodiments, the attachment member 118 may be configured to receive/engage a corresponding surface of the second endoscopic instrument 150 (e.g., a handle assembly 154 of the second endoscopic instrument) in a snap-lock configuration such that the second endoscopic instrument 150 may move laterally (e.g., back-and-forth) along/relative to along a longitudinal axis of the main body 110.

In one embodiment, a first support structure 120 may be attached to or integrally formed with the main body 110 at or near a proximal end of the first lumen 114 and a second support structure 122 may be attached to or integrally formed with the main body 110 at or near the distal end of the first and second lumens 114, 116. In various embodiments, the first and second support structures 120, 122 may be configured to eliminate, minimize or reduce bending or kinking of the first endoscopic instrument 140 (e.g., radial ultrasound probe) extending through the contiguous first lumens 114, 115, which may impair or otherwise negatively affect ultrasound image quality. For example, the first and second support structures 120, 122 may limit the extent to which the first endoscopic instrument 140 bends or flexes within the main body 110 and/or the proximal portion of the dual-lumen catheter 112, e.g., to allow the requisite high-speed rotation of the radial ultrasound probe throughout the full length of the dual-lumen catheter 112. In addition, or alternatively, the first and second support structures 120, 122 may provide a dampening or insulating function to prevent, minimize or eliminate outside forces acting on the main body 110, e.g., contact by other medical devices and/or the hand(s) of a medical professional operating the modular assembly, from propagating through the main body 110 and interfering with or otherwise corrupting the ultrasound signal. In various additional embodiments, the first and second support structures 120, 122 may provide a leak-proof seal at the proximal and distal ends of the first lumen 114 of the main body 110 such that fluid flushed through the contiguous first lumens 114, 115 of the main body 110 and dual-lumen catheter 112 (e.g., to remove air from within the first lumens 114, 115 and/or improve propagation of the ultrasound signal with the pulmonary passageway) does not leak into the patient and/or the contiguous second lumens 116, 117 of the main body 110 and dual-lumen catheter 112.

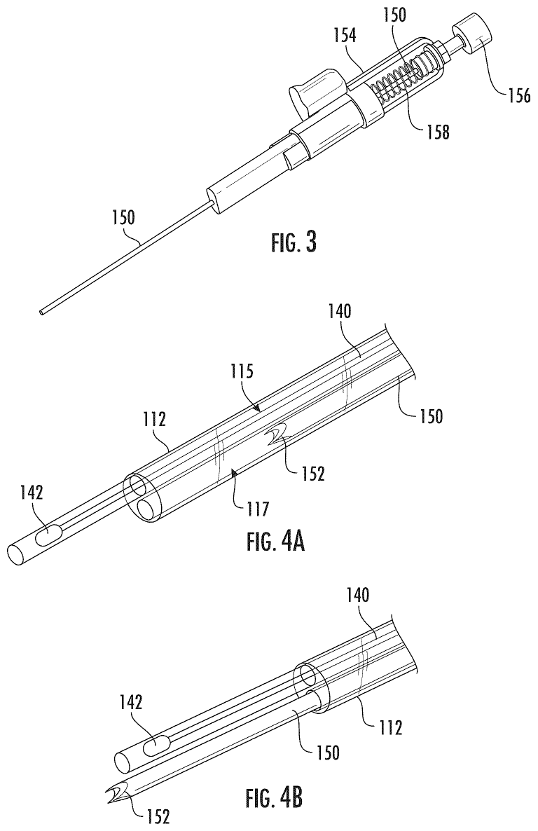

Referring to FIG. 2, in one embodiment, a system 200 of the present disclosure may include a first endoscopic instrument 140 (e.g., radial ultrasound probe) disposed within the first lumens 114, 115 of the main body 110 and dual-lumen catheter 112 such that an ultrasound transducer 142 disposed at a distal end of the first endoscopic instrument 140 extends beyond a distal end of the dual-lumen catheter 112 (FIG. 4A). In various additional embodiments, a second endoscopic instrument 150 (e.g., elongate biopsy needle) may be slidably disposed within the second lumens 116, 117 of the main body 110 and dual-lumen catheter 112 to move a sharpened distal end 152 of the second endoscopic instrument 150 between a first position, e.g., proximally retracted, shielded or protected within the second lumen 117 (FIG. 4A), and a second position, e.g., extended distally beyond or exposed from within the second lumen 117 (FIG. 4B).

In one embodiment, a handle assembly 154 (FIG. 3) of the second endoscopic instrument 150 may be engaged with the attachment member 118 of the main body 110 in a snap-lock configuration. In various embodiments, a proximal portion of the second endoscopic instrument 150 may extend through the handle assembly 154 such that a proximal end of the second endoscopic instrument 150 is attached to a plunger 156 extending beyond a proximal end of the handle assembly 154. A spring 158 may be disposed around an outer circumference of a portion of the second endoscopic instrument 150 such that the plunger 156 may move between a first position (e.g., non-depressed, non-deployed) and second position (e.g., depressed, deployed, etc.).

Referring to FIGS. 4A-4C, in various embodiments, the handle assembly 154 may move laterally (e.g., slide back-and-forth) along a longitudinal axis of the main body 110 of the modular assembly 100 between a proximally retracted position with the sharpened distal end 152 of the second endoscopic instrument 150 in the first position (FIG. 4A) and a distally extended position with the sharpened distal end 152 of the second endoscopic instrument in the second position (FIG. 4B). In various additional embodiments, with the handle assembly 154 in the distally extended position and the sharpened distal end 152 of the second endoscopic instrument 150 in the second position, the plunger 156 may be moved from the first position to the second position to further distally advance the sharpened distal end 152 of the second endoscopic instrument 150 relative to the ultrasound transducer 142 (FIG. 4C).

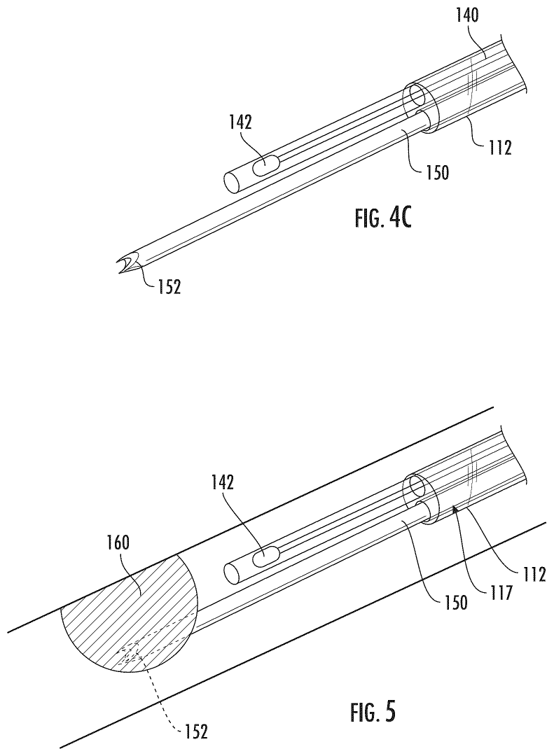

Referring to FIG. 5, in use and by way of example, a bronchoscope (not shown) may be advanced into a pulmonary passage of a patient (e.g., through the esophagus). A dual-lumen catheter 112 of the modular assembly 100 may then be introduced into a working channel of the bronchoscope and distally advanced through the working channel, e.g., by a medical professional holding the main body 110 of the modular assembly 100. The dual-lumen catheter 112 may be distally advanced beyond the distal end of the bronchoscope and into a peripheral region of the lung (e.g., where early cancerous pulmonary nodules tend to form) under radial ultrasound direct imaging guidance provided by the ultrasound transducer 142 extending beyond the distal end of the dual-lumen catheter 112 to identify a first pulmonary nodule 160. The handle assembly 154 of the second endoscopic instrument 150 may then be moved laterally along a longitudinal axis of the main body 110 of the modular assembly 100 from a proximally retracted position to a distally extended position to expose the sharpened distal end 152 of the second endoscopic instrument 150 from within the second lumen 117 of the dual-lumen catheter 112. In various embodiments, the sharpened distal end 152 of the second endoscopic instrument 150 may be substantially adjacent to or extend slightly distally beyond the ultrasound transducer 142 such that the pulmonary nodule 160 and sharpened distal end 152 may be imaged simultaneously. In various embodiments, the main body 110 of the modular assembly 100 may be rotated and/or distally advanced to position/align the sharpened distal end 152 of the second endoscopic instrument 150 with the pulmonary nodule 160. The plunger 156 may then be repeatedly moved between the first and second positions to extend/advance the sharpened distal end 152 of the second endoscopic instrument 150 into the pulmonary nodule 160. In various embodiments, the plunger 156 may be depressed multiple (e.g., 12-15) times to ensure that sufficient volume and location of biopsy sample of the pulmonary nodule 160 is acquired within the lumen of the sharpened distal end 152 of the second endoscopic instrument 150 for diagnostic analysis.

In various embodiments, the handle assembly 154 may then be detached from the attachment member 118 of the main body 110 and the second endoscopic instrument 150 withdrawn/retracted through the contiguous second lumens 116, 117 of the main body 110 and dual-lumen catheter 112 while the dual-lumen catheter 112 remains disposed within the working channel of the bronchoscope. The biopsy sample may then be ejected from within the lumen of the second endoscopic instrument 150 for diagnostic analysis and a second endoscopic instrument 150 (e.g., the same second endoscopic instrument or a different/second endoscopic instrument) reinserted/inserted through the contiguous second lumens 116, 117 such that the sharpened distal end 152 returns to the first position (FIG. 4A) within the dual-lumen catheter 112 and with the ultrasound transducer 142 having remained adjacent to and visualizing the first pulmonary nodule 160. In various embodiments, an additional biopsy sample may be acquired from the first pulmonary nodule 160 by repeating the steps outlined above. Alternatively, the dual-lumen catheter 112 may be repositioned within the peripheral region of the lung (e.g., advanced to a more distal peripheral region or a different peripheral branch) to visualize a second pulmonary nodule (not shown). The handle assembly 154 and plunger 156 may then be actuated, and the sharpened distal end 152 of the second endoscopic instrument 150 repositioned and/or rotated, as discussed above to obtain a biopsy sample from the second pulmonary nodule.

In various embodiments, the modular assembly 100 and system 200 described above may provide a variety of advantages as compared to conventional assemblies and systems. By way of non-limiting example, a first advantage may be provided by the second endoscopic instrument 150 extending through the second lumen 117 of the dual-lumen catheter 112, which may provide columnar support to the dual-lumen catheter 112 to protect the first endoscopic instrument 140 from excessive bending, twisting or kinking within the narrow and tortuous pulmonary passages. A second advantage may be provided by the sharpened distal end 152 of the second endoscopic instrument 150 being maintained within the dual-lumen catheter 112 as a pulmonary nodule is imaged, thereby allowing a biopsy sample to be acquired upon imaging without requiring a device exchange. A third advantage may be provided by the plunger and spring of the handle assembly, which may allow a medical professional to actuate the second endoscopic instrument using a single hand, e.g., to obtain a sufficient biopsy sample from the pulmonary nodule. A fourth advantage may be provided by the side-by-side configuration of the first and second lumens 115, 117 of the dual-lumen catheter 112, which may allow a medical professional to rotate the main body 110 of the modular assembly 100 to align the sharpened distal end 152 of the second endoscopic instrument 150 with an eccentric pulmonary nodule while maintaining visualization of the pulmonary nodule (e.g., as the second endoscopic instrument 150 rotates around/relative to an axis of the first endoscopic instrument 140). A fourth advantage may be provided by the main body 110 of the modular assembly 100 being configured for single-user control or multiple-user control. For example, multiple-user control may allow a first medical professional (e.g., a physician) to position the first endoscopic instrument 140 adjacent to a target pulmonary nodule and a second medical professional (e.g., a medical assistant) to manipulate the second endoscopic instrument 150 (e.g., acquire the biopsy sample and remove the second endoscopic instrument through the working channel) at the instruction of the first medical professional. Alternatively, single-user control may allow a medical professional to manipulate the first endoscopic instrument 140 with one hand and manipulate the second endoscopic instrument 150 with the other hand.

In various embodiments, the second endoscopic instrument 150 of the present disclosure is not limited to a biopsy needle, but may include a variety of medical instruments configured to manipulate a target tissue within a body passage, including, for example, electrocautery knives, clips, brushes, scissors, graspers, forceps, laser ablative elements, etc. Other peripheral pulmonary nodule visualization and/or biopsy techniques, features, and/or components that may be used herein are disclosed in U.S. Non-Provisional Patent Application titled “Apparatus to Provide an Adjustable Mechanism for Radial Ultrasound Port and Flush Port”, attorney docket number 8150.0600, filed even date herewith, the entirety of which is incorporated herein by reference and/or U.S. Non-Provisional Patent Application titled “Medical Imaging Devices, Systems, and Methods”, attorney docket number 8150.0746, filed even date herewith, the entirety of which is incorporated herein by reference.

All of the devices and/or methods disclosed and claimed herein can be made and executed without undue experimentation in light of the present disclosure. While the devices and methods of this disclosure have been described in terms of preferred embodiments, it may be apparent to those of skill in the art that variations can be applied to the devices and/or methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

What is claimed is:

1. A modular assembly, comprising:
a main body;
a dual-lumen catheter attached to a distal end of the main body;
a first lumen extending through the main body, the first lumen contiguous with a first lumen of the dual-lumen catheter;
a second lumen extending through the main body, the second lumen contiguous with a second lumen of the dual-lumen catheter;
an attachment member formed within the main body at a proximal end of the second lumen;
a first support structure attached to a proximal end of the main body at a proximal end of the first lumen; and
a second support structure attached to a distal end of the main body at a distal end of the first lumen, wherein the first and second support structures provide a leak-proof seal for the first lumen.

2. The modular assembly of claim 1, wherein the first lumens of the main body and the dual-lumen catheter are configured to receive a first endoscopic instrument therethrough, and wherein the second lumens of the main body and the dual-lumen catheter are configured to receive a second endoscopic instrument therethrough.

3. The modular assembly of claim 2, wherein the attachment member is configured to receive a handle assembly of the second endoscopic instrument in a snap-lock configuration.

4. A system, comprising:
a modular assembly comprising:
a main body,
a dual-lumen catheter attached to a distal end of the main body,
a first lumen extending through the main body, the first lumen contiguous with a first lumen of the dual-lumen catheter,
a second lumen extending through the main body, the second lumen contiguous with a second lumen of the dual-lumen catheter,
an attachment member formed within the main body at a proximal end of the second lumen.
a first support structure attached to a proximal end of the main body at a proximal end of the first lumen, and
a second support structure attached to a distal end of the main body at a distal end of the first lumen, wherein the first and second support structures provide a leak-proof seal for the first lumen;
a first endoscopic instrument extending through the first lumens of the main body and the dual-lumen catheter; and
a second endoscopic instrument extending through the second lumens of the main body and dual-lumen catheter.

5. The system of claim 4, wherein the first endoscopic instrument is a radial ultrasound probe and the second endoscopic instrument is a biopsy needle.

6. The system of claim 4, wherein the second endoscopic instrument is configured to move laterally between a first and second position within the dual-lumen catheter.

7. The system of any of claims 4-5, wherein the attachment member is configured to receive a handle assembly of the second endoscopic instrument in a snap-lock configuration.

8. The system of claim 7, wherein the handle assembly is configured to move laterally along the main body of the modular assembly.

9. The system of claim 8, wherein the handle assembly is configured move from a first position in which a distal end of the second endoscopic instrument is disposed within the second lumen of the dual-lumen catheter, and a second position in which the distal end of the second endoscopic instrument extends beyond a distal end of the dual-lumen catheter.

10. The system of any of claim 9, wherein the handle assembly includes a plunger configured to move the second endoscopic instrument between the first and second position.

11. The system of any of claims 4-10, wherein the dual-lumen catheter is configured to extend through a working channel of a bronchoscope.

12. A modular assembly, comprising:
a main body;
a dual-lumen catheter attached to a distal end of the main body, the dual-lumen catheter having a shaft having a first lumen and a second lumen disposed within the shaft;
a first lumen extending through the main body, the first lumen contiguous with the first lumen of the dual-lumen catheter;
a second lumen extending through the main body, the second lumen contiguous with the second lumen of the dual-lumen catheter; and
an attachment member formed within the main body at a proximal end of the second lumen,
wherein the first lumens of the main body and the dual-lumen catheter are configured to receive a first endoscopic instrument therethrough, and wherein the second lumens of the main body and the dual-lumen catheter are configured to receive a second endoscopic instrument therethrough, and
wherein the attachment member is configured to receive a handle assembly of the second endoscopic instrument in a snap-lock configuration.

13. The modular assembly of claim 12, further comprising a first support structure attached to a proximal end of the main body at a proximal end of the first lumen.

14. The modular assembly of claim 13, further comprising a second support structure attached to the distal end of the main body at a distal end of the first lumen.

15. A system, comprising:
a modular assembly comprising:
a main body,
a dual-lumen catheter attached to a distal end of the main body, the dual-lumen catheter having a shaft having a first lumen and a second lumen disposed within the shaft,
a first lumen extending through the main body, the first lumen contiguous with the first lumen of the dual-lumen catheter,
a second lumen extending through the main body, the second lumen contiguous with the second lumen of the dual-lumen catheter, and
an attachment member formed within the main body at a proximal end of the second lumen;
a first endoscopic instrument extending through the first lumens of the main body and the dual-lumen catheter; and
a second endoscopic instrument extending through the second lumens of the main body and dual-lumen catheter,
wherein the second endoscopic instrument comprises a handle assembly, and wherein the attachment member is configured to receive the handle assembly of the second endoscopic instrument in a snap-lock configuration.

16. The system of claim 15, wherein the first endoscopic instrument is a radial ultrasound probe and the second endoscopic instrument is a biopsy needle.

17. The system of claim 15, wherein the second endoscopic instrument is configured to move laterally between a first and second position within the dual-lumen catheter.

18. The system of claim 15, wherein the handle assembly is configured to move laterally along the main body of the modular assembly.

19. The system of claim 18, wherein the handle assembly is configured to move from a first position in which a distal end of the second endoscopic instrument is disposed within the second lumen of the dual-lumen catheter, and a second position in which the distal end of the second endoscopic instrument extends beyond a distal end of the dual-lumen catheter.

20. The system of claim 19, wherein the handle assembly includes a plunger configured to move the second endoscopic instrument between the first and second position.

21. The system of claim 19, further comprising a first support structure attached to a proximal end of the main body at a proximal end of the first lumen.

22. The system of claim 21, further comprising a second support structure attached to the distal end of the main body at a distal end of the first lumen.
